(19) United States
(12) Patent Application Publication
(10) Pub. No.: US 2013/0090546 A1
(43) Pub. Date: Apr. 11, 2013

(54) SPINAL CORD FUNCTION ASSESSMENT

(71) Applicant: Patrick W. STROMAN, Kingston (CA)

(72) Inventor: Patrick W. STROMAN, Kingston (CA)

(21) Appl. No.: 13/269,300

(22) Filed: Oct. 7, 2011

(51) Int. Cl. A61B 5/055; A61B 5/103

(52) U.S. Cl. 600/411; 600/555

(57) ABSTRACT

A device, system and methods are described for stimulating multiple dermatones on a body in accordance with a stimulation pattern. Magnetic resonance images are captured during the application of the stimulation pattern and the captured images are processed. The images are processed using information about the applied stimulation pattern to assess spinal cord function.

### FIELD OF THE INVENTION

The current application relates generally to the assessment of spinal cord function and in particular to the assessment of spinal cord function using functional magnetic resonance imaging.

### BACKGROUND

In assessing damage to the spinal cord by injury or disease, knowledge of spinal cord function is required. Current standard clinical tests for spinal cord function include pin-prick tests across dermatomes and motor tests of various muscle groups. Electrophysiological tests, involving stimulation of cortical areas and recording of motor and sensory evoked potentials, are also used to reveal functional connections. Other assessments currently in clinical use include surveys of the patient's abilities and quality-of-life factors. The information generated from these methods is limited and do not reveal information about spinal cord function below the most rostral point of damage. Further the thoracic regions of the cord are difficult to assess, or the assessments are subjective.

Functional magnetic resonance imaging (fMRI or functional MRI) has been shown to provide information missed by current clinical assessments. For instance, fMRI of the human spinal cord can demonstrate activity caudal to sites of spinal cord injury at any level of the cord, the effects of multiple sclerosis and peripheral nerve damage, and can demonstrate spinal cord activity related to sexual function. fMRI imaging has been used to see the activity associated with a stimulation device applied to a dermatone. However, the stimulation has been limited to a single dermatone.

### SUMMARY

In accordance with the disclosure there is provided a method of assessing spinal cord function comprising applying a respective stimulation pattern of a plurality of linearly independent stimulation patterns to a respective stimulation device of plurality of stimulation devices each attached to a respective dermatome of a patient; capturing a plurality of functional magnetic resonance (MR) images during the application of the plurality of linearly independent stimulation patterns; and analyzing the plurality of captured functional MR images based on the applied linearly independent stimulation patterns to assess spinal cord function.

In accordance with the disclosure there is further provided a system for assessing spinal cord function comprising a plurality of stimulation devices for attachment to respective dermatomes of a patient; a control device for applying a respective stimulation pattern of a plurality of linearly independent stimulation patterns to a respective stimulation device; and an analysis device for analyzing a plurality of functional magnetic resonance (MR) images of the patient captured during the application of the stimulation patterns to assess spinal cord function, the analysis of the plurality of functional MR images based on the applied linearly independent stimulation patterns.

In accordance with the disclosure there is further provided a control device for use in assessing spinal cord function comprising: an interface for connecting the control device to a plurality of stimulation devices; a microcontroller for applying a respective stimulation pattern of a plurality of linearly independent stimulation patterns to a respective stimulation device when connected to the interface.

In accordance with the disclosure there is further provided a kit comprising: a control device for use in assessing spinal cord function comprising: an interface for connecting the control device to a plurality of stimulation devices; a microcontroller for applying a respective stimulation pattern of a plurality of linearly independent stimulation patterns to a respective stimulation device when connected to the interface; and a plurality of stimulation devices for attaching to a patient and connecting to the device interface of the control device.

### BRIEF DESCRIPTION OF THE DRAWINGS

Embodiments are described herein with references to the appended drawings, in which:

FIG. 1 depicts in a block diagram, a system for assessing spinal cord function;

FIG. 2 depicts in a flow diagram a method of assessing spinal cord function;

FIG. 3A depicts in a top view a block diagram of a stimulation device that may be used with the system for assessing spinal cord function;

FIG. 3B depicts in a side view a block diagram of a stimulation device that may be used with the system for assessing spinal cord function;

FIG. 4 depicts in a time line a stimulation pattern;

FIG. 5 depicts in a time line a further stimulation pattern;

FIG. 6 depicts in a schematic components of a control device that may be used with the system for assessing spinal cord function;

FIG. 7 depicts in a flow diagram a method of stimulating a controlling a plurality of thermodes; and

FIG. 8 depicts in a flow diagram a method of analyzing images to assess spinal cord function.

### DETAILED DESCRIPTION

Numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

In assessing spinal cord functionality it is desirable to observe activity from dermatones above and below the area under observation, as well as to the left and right of the area. Functional magnetic resonance imaging (fMRI) has been used to visualize activity associated with stimulation applied to a dermatone. However, the stimulation has been limited to a single stimulation device, which makes its use in assessing spinal cord function near a location of possible damage of limited value. Activity in the spinal cord is modulated by descending input from the brainstem and cortex and can depend upon factors of awareness, alertness, and attention as well as control of motor reflex responses. As such, the same stimulation applied to a dermatone at different times may result in different activity levels. Similarly, the same stimulation applied to different dermatones at different times may result in different observed activity levels, making it difficult to relate the observed activity levels from stimulating different dermatones using a single stimulation. When the stimulation is applied under different conditions, such as would be required when using a single stimulation device to stimulate multiple dermatones, it is difficult to determine if the modulating factors affecting the response to the stimulation are the same, making it difficult to compare the activity levels of the spinal cord function from multiple dermatones using a single stimulation device.

As described further herein, applying a plurality of stimulation devices to a patient simultaneously and capturing a time series of images using fMRI can provide information about spinal cord function associated with different dermatones that can be correlated together to provide information about spinal cord function in an area under assessment.

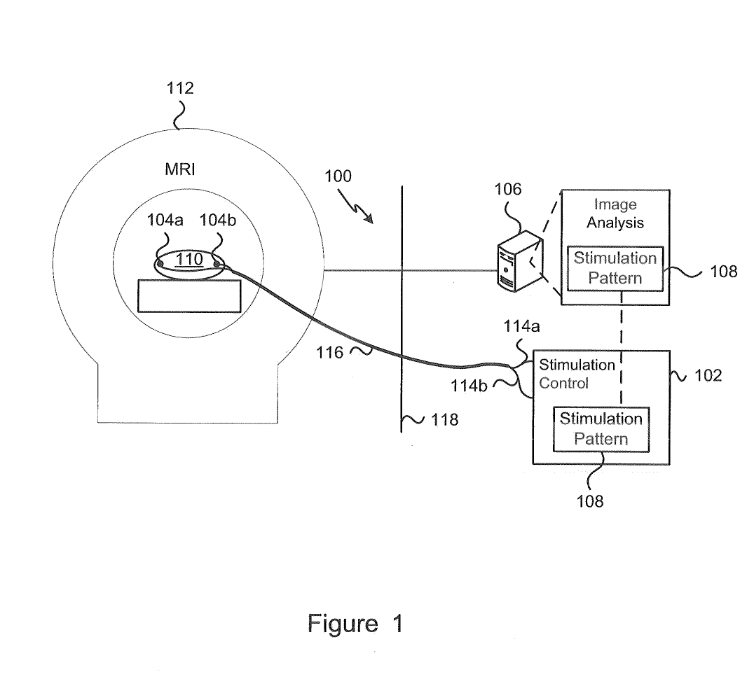

FIG. 1 depicts in a block diagram, a system for assessing spinal cord function. The system 100 comprises a control device 102 connected to a plurality of stimulation devices 104a, 104b (referred to collectively as stimulation devices 104) and an analysis device 106. The analysis device may be provided by a separate computing device or may be incorporated into the control functionality of a functional magnetic resonance imaging (fMRI) machine 112. As described further herein, the control device 102 controls the stimulation devices 104 based on respective stimulation patterns 108. The stimulation devices 104 are placed on a patient's body 110. Each of the stimulation devices 104 may be connected to the control device 102 by a respective cable 114a, 114b (referred to collectively as cables 114). The individual cables 114 may be gathered together into a bundle by a sheath 116. The sheath 116 may provide a degree of shielding from magnetic fields of the MRI system. The sheath may be provided by a sheath or jacket made from an electrical conductor. Additionally, the sheath 116 may be electrically connected to ground. The MRI system 112 captures a time series of images of the patient 110, including an area surrounding a portion of the spinal cord, while the stimulation patterns are applied to the stimulation devices 104. The captured images are provided to the analysis device 106, which processes the images to assess the spinal cord function. The analysis device 106 includes the stimulation patterns 108, or information about the stimulation patterns 108, used by the control device 102, which the analysis device 106 uses in processing the images. Both the control device 102 and the analysis device 106 may be physically separated from the MRI system by a wall 118 in order to contain the magnetic fields of the MRI system. Although described as assessing spinal cord function, it will be appreciated that the system 100 may provide information to a professional that can be used to aide the professional in assessing spinal cord function.

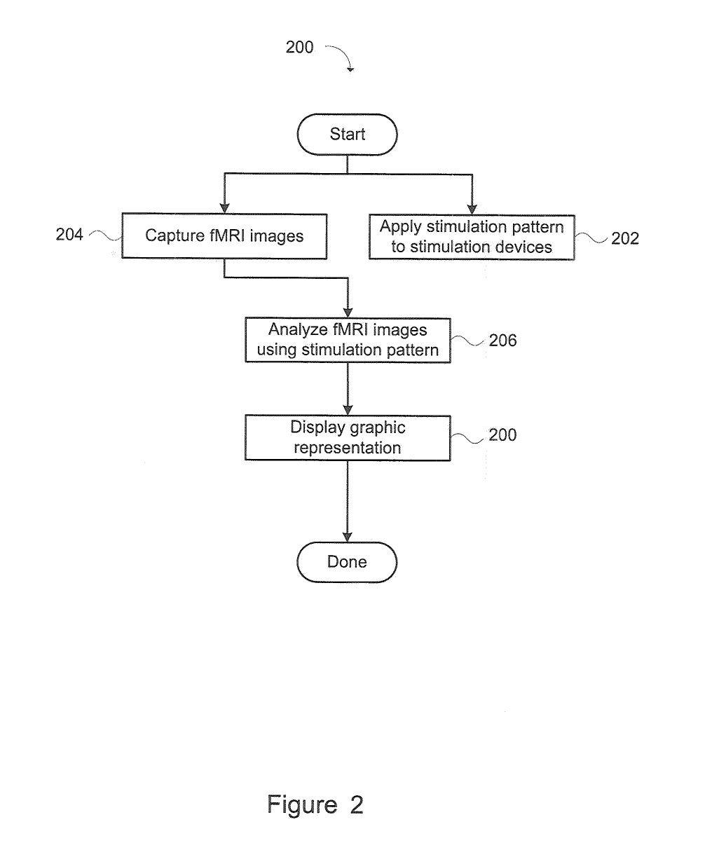

FIG. 2 depicts in a flow diagram a method of assessing spinal cord function. When the method 200 starts, stimulation patterns are applied to a plurality of stimulation devices (202). The stimulation patterns are linearly independent. Respective stimulation patterns are applied to each stimulation device, in order to allow changes in activity levels in the captured images to be associated with an individual stimulation device attached to a particular dermatone. While the stimulation devices are being stimulated in accordance with the respective stimulation patterns, a plurality of images is captured (204) by an MRI system. The images may be captured at a particular frequency, for example, an image may be captured every 9 seconds. It will be appreciated that the images may be captured at various frequencies depending on the configuration of the MRI system. Once the stimulation patterns are completed, the time series of images captured during the application of the stimulation patterns is processed (206). The processing may identify the activity levels associated with the different dermatones being stimulated in order to assess the spinal cord function in the area being observed. The image analysis uses the stimulation patterns that were applied to the stimulation devices in order to assess the spinal cord activity resultant from the applied stimulation. The processing detects, or attempts to detect, in the images a consistent response pattern over time that matches one of the applied stimulation patterns. A graphic representation of the image analysis can be generated and displayed (208). The graphic representation may be used as an aid in assessing the spinal cord function of the patient.

Two stimulation devices 104 are depicted in FIG. 1; however four or more stimulation devices may be placed on the patient. If four stimulation devices are used, they are attached to the patient on dermatones above and to the left, above and to the right, below and to the left as well as below and to the right of an area of the spinal cord being assessed. Locations of dermatones, and the location of the spinal cord where activity associated with them occurs, are well known and is not discussed further herein.

The area of the spinal cord being assessed may be associated with a spinal cord injury, or spinal cord disease such as multiple sclerosis. The activity in the neural tissue that results from the stimulation of the various dermatones the stimulation devices are attached to is captured in the time series of images captured by the MRI system. The analysis of the images using the stimulation pattern allows the activity of the spinal cord associated with the stimulated dermatones to be analyzed and the spinal cord function in the surrounding area to be assessed.

As described further herein, the stimulation devices 104 may be provided by thermodes, which can be attached to the patient's skin and caused to heat or cool to a particular temperature or temperature range. Although thermodes are described, other stimulation devices that provide tactile stimulation may be used. For example a stimulation device that provides stimulation through pressurized air, or through a pin-prick may be used.

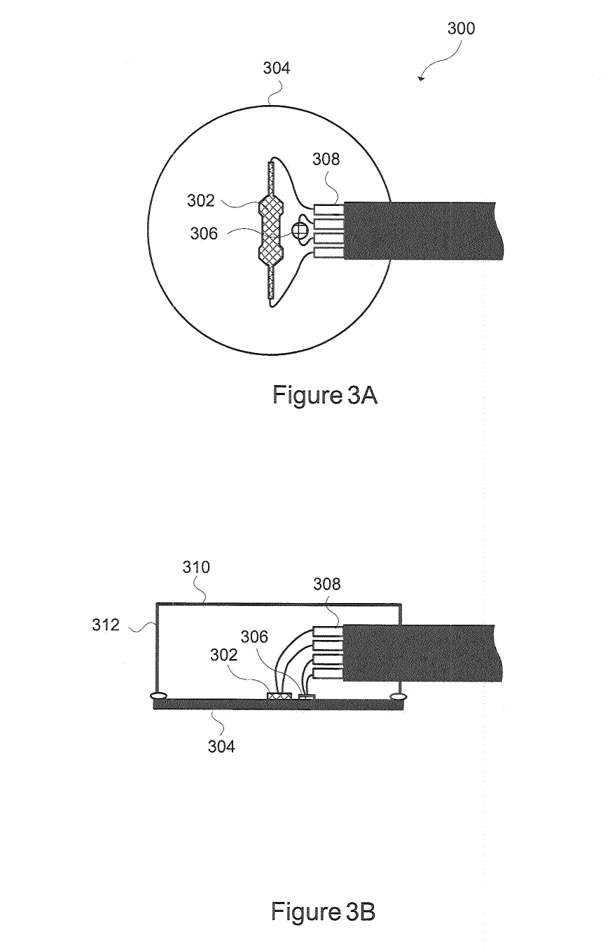

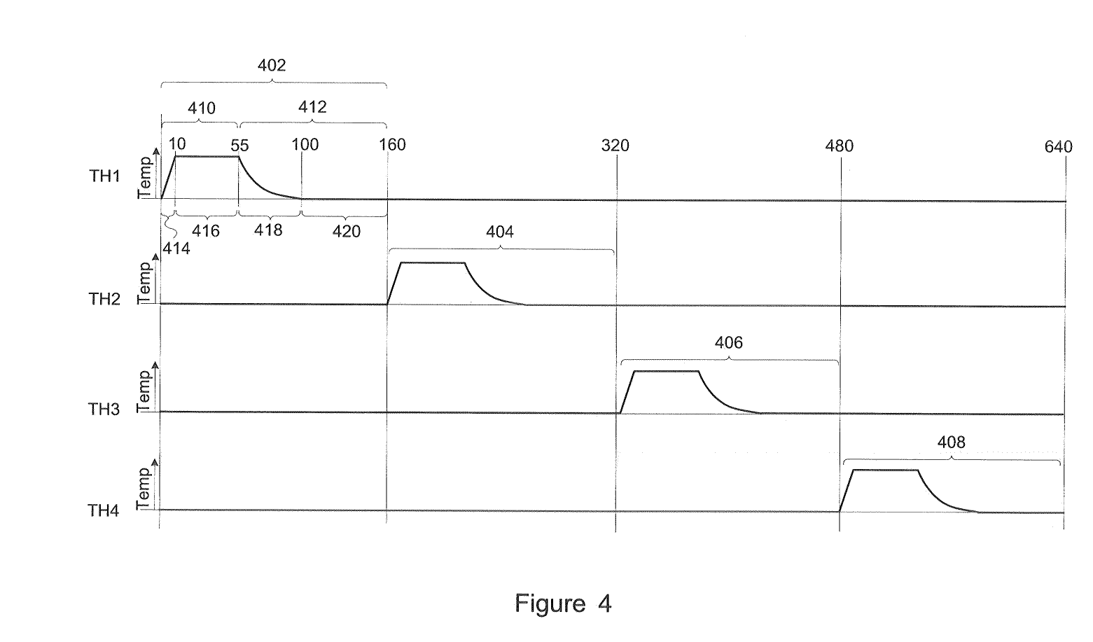

FIGS. 3A and 3B depict, in top and side views respectively, a diagram of a stimulation device that may be used with the system for assessing spinal cord function. The stimulation device 300 is a thermode. The thermode 300 comprises a resistor 302 that is attached to a thermal plate 304, which may be made from a good thermal conductor, for example copper. The resistor 302 heats as current is passed through it under control of the stimulation control device described above. The heat is transferred to the thermal plate 304, which is attached to the patient's skin. A thermistor 306 is also attached to the thermal plate 304 and is used by the stimulation control device to measure the temperature of the thermal plate 304 in order to control the thermode 300. Both the resistor 302 and the thermistor 306 may be connected to the control device via appropriate wires 308. The connecting wires 308 may be provided by twisted pair cables, such as CAT 5 cabling. The connecting wires 308 may also be shielded by a metal sheath, or they may be shielded by the sheath around the group of connecting wires from the various stimulation devices.

The thermode 300 may also comprise an electrically conducting housing 310 enclosing the resistor 302 and thermistor 306 in order to further protect the components and electrical signals from the magnetic fields of the MRI system. The housing 310 may be connected to the grounded sheath of the connecting wires if used. The housing 310 may be cylindrical in shape and be connected to the thermal plate 304 using an adhesive 312.

Although the thermode 300 is described as using a resistor 302 for the heating element other devices may be used, such as a peltier device.

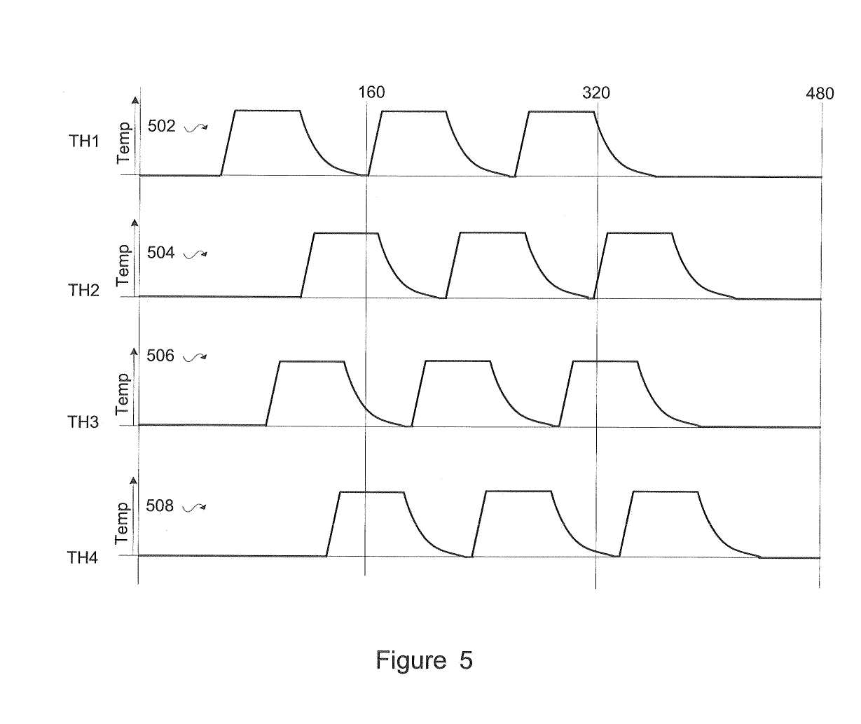

FIG. 4 depicts in a time line stimulation patterns that may be applied to four stimulation devices. The stimulation patterns of FIG. 4 are assumed to be associated with thermode stimulation devices. The stimulation patterns 402, 404, 406, 408 are depicted as having an on period which is sequentially applied to the associated thermode TH1, TH2, TH3, TH4 attached to respective dermatones. For example the first thermode TH1 may be attached to the right hand, the second thermode TH2 may be attached to the left hand, the third thermode TH3 may be attached to the right shoulder and the fourth thermode TH4 may be attached to the left shoulder. As depicted, each stimulation pattern may be the same pattern. It is contemplated that the stimulation patterns 402, 404, 406, 408 could be different from each other.

Each of stimulation patterns 402, 404, 406, 408 depicted in FIG. 4 comprises a plurality of areas. Each of the base stimulation patterns comprises an ‘on’ period 410, during which the associated thermode is being heated, and an ‘off’ period 412, during which the associated thermode is not being heated. The on period 410 comprises a heating period 414 during which a current is applied to the thermode in order to heat it, and a hold period during which the heating of the thermode is moderated in order to maintain the temperature of the thermode. The temperature of the thermode may be maintained at a preset temperature, such as 44° C. The hold period may last for a set period of time such as 45 seconds. Alternatively, the on period 410, that is the combined heat and hold periods, may last for a set period of time such as 45 seconds.

The off period 412 begins following the on period 410, that is the combined heat and hold periods. The off period 412 comprises a cooling period 416 during which no current flows through the thermode and as such, the thermode cools down. The cooling period 416 may last for a set period of time, such as 45 seconds. Following the cooling period 416 there is a rest period 418. During the rest period, the cooled off thermode remains off for a set period of time, such as 60 seconds.

As depicted in FIG. 4, once an associated stimulation pattern has been applied to one thermode, the on period of the stimulation pattern associated with the next thermode occurs. As depicted in FIG. 4, each thermode has an associated stimulation pattern applied with only one stimulation pattern being applied to a thermode at a time, or at least having one on period of the stimulation patterns occurring at once. The stimulation patterns of all of the thermodes may be repeated a number of times. The stimulation patterns applied to the thermode can be controlled by a control device that the thermodes are connected to.

As depicted in FIG. 4, the application of the stimulation patterns may take approximately 640 seconds to complete. If the stimulation patterns are repeated numerous times to provide additional data points, the application could take over 30 minutes, if three cycles of the stimulation patterns are applied. It may be desirable to provide stimulation patterns that requires less time to complete, since it may be difficult for a patient to lie still for 30 minutes as required for fMRI as well as to limit the time required to use the MRI system, which may be difficult to arrange or schedule.

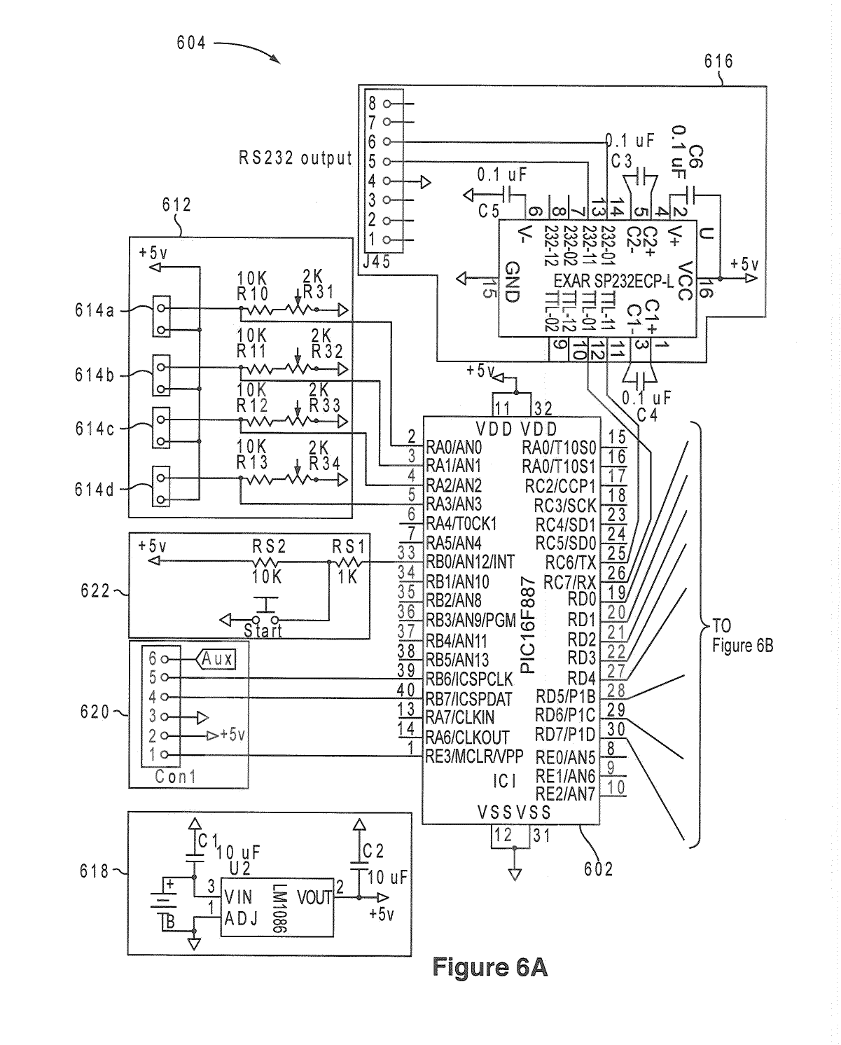

FIG. 5 depicts in a time line further stimulation patterns that may be applied to four stimulation devices. As depicted, each of the stimulation patterns 502, 504, 506, 508, is associated with a respective stimulation device. As depicted in FIG. 5, each stimulation pattern 502, 504, 506, 508 comprises a plurality of on periods during which the associated thermode is heated, followed by an off period during which the thermode is allowed to cool down. The length of time of the on and off periods may vary. For example the on period may last for approximately 45 seconds, during which time the associated thermode is heated up to a target temperature and held there. Following the on period, the thermode may be allowed to passively cool for a cool down and rest period which may last for, for example, 60 seconds. As depicted, each stimulation pattern may heat and cool the associated thermode three times, although it is contemplated that fewer or more heating and cooling cycles could be applied.

As depicted in FIG. 5, the on periods of the stimulation patterns may overlap. By overlapping the on periods of the stimulation patterns, the time required to complete the whole stimulation pattern is reduced. In order to ensure that the activity associated with a particular stimulation device can be differentiated from the activity of other stimulation devices, the stimulation patterns are linearly independent. As depicted in FIG. 5, the overall stimulation pattern, which includes three heating and cooling cycles for each of the four thermodes takes approximately 400 seconds to complete.

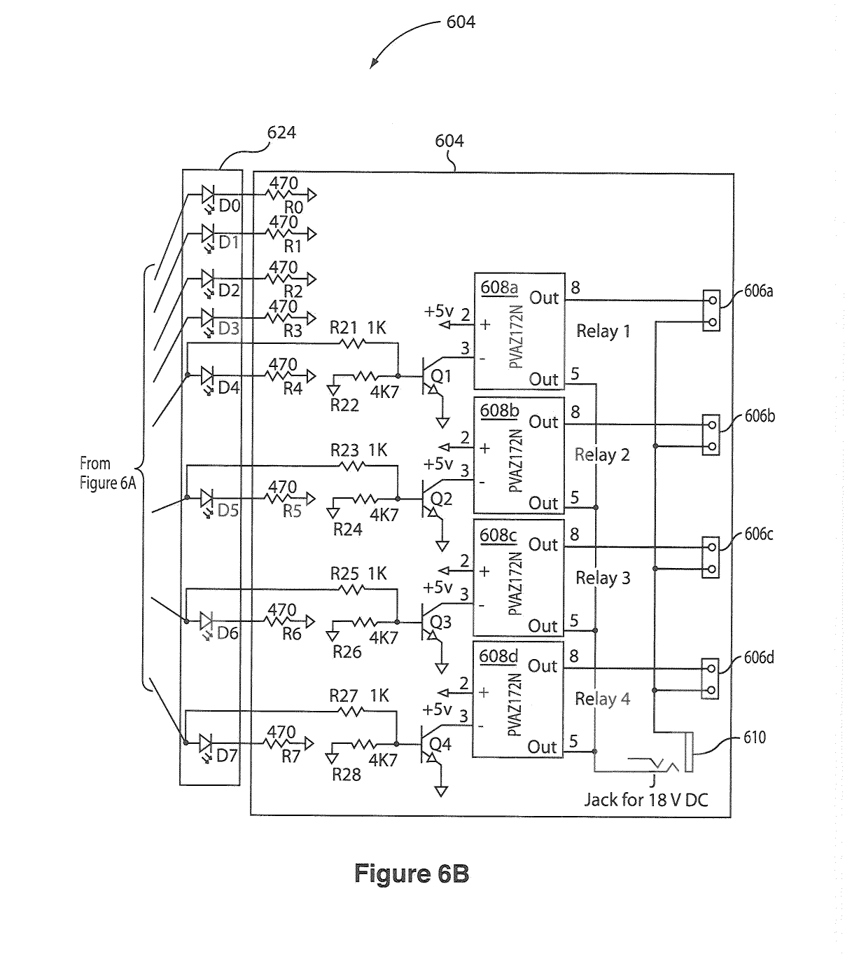

FIG. 6 depicts in a schematic components of a control device that may be used to control a plurality of thermodes according to a stimulation pattern. The device 600 comprises a microcontroller 602 that is programmed to control apply respective stimulation patterns to connected thermodes. The microcontroller 602 is depicted as a Microchip PIC® 8-bit microcontroller, in particular a PIC 16F887. It is contemplated that the microcontroller 602 may be provided by various other microcontrollers or processors. The control device 600 further comprises an output section 604 for providing current to connected thermodes. The output section 604 comprises a plurality of output connections 606a, 606b, 606c, 606d for connecting to the resistor of respective thermodes. Each of the output connections can be driven by a respective relay 608a, 608b, 608c, 608d that connects the thermodes to a power supply, which is depicted as a being provided by a connection 610 to an external 18 volt direct current source. Each of the relays 608a, 608b, 608c, 608d is controlled by a respective transistor and signal output from the microcontroller 602. When the microcontroller 602 asserts the associated signal, the associated relay is closed and the 18 volt signal is applied across the resistor of the thermode, causing it to heat. Although the output to each thermode is depicted as being controlled by a relay, it is contemplated that other devices, such as transistors, may be used.

The control device 600 further comprises an input section 612 that provides for temperature feedback from the thermsistors of the thermodes. The output section 612 comprises a plurality of input connections 614a, 614b, 614c, 614d for connecting to the thermistors of the thermodes. The input connections are connected to analog inputs of the microcontroller 602. Each input connection applies a voltage across the connected thermistor, which varies its resistance based on its temperature. The microcontroller senses the voltage across a voltage divider circuit and can convert the sensed analog voltage to an associated temperature. Although analog signals are depicted as being provided to the microcontroller, it is contemplated that a separate analog to digital converter may be used in order to provide a digital input representing the temperature.

The control device 600 may further comprise a communication section 616. The communication section 616 is depicted as being provided by an Exar SP232ECP-L which is an RS 232 interface. It is contemplated that other interfaces may be provided The communication section 616 enables bi-directional communication with an external device. The communication section 616 may be used to communicate various information, including stimulation patterns and the sensed temperatures, to an external device. Further, the communication section 616 may be used to provide different stimulation patterns to the control device from an external device.

The control device 600 may include a voltage regulator 618, depicted as being provided by an LM1086 which can provide a regulated 5 volt output from an unregulated power source. The regulated 5 volt signal can be distributed to other components of the control device 600. Although not depicted in FIG. 6, a power switch may be included for turning on or off power to the device 600.

The control device may further comprise a programming interface 620 for coupling the microcontroller 602 to an external device. The programming interface 620 can be used to program the microcontroller 602. The programming interface 620 may also provide a 5 volt signal that can be used in stead of the signal provided by the voltage regulator 618.

The control device 600 may further comprises a start switch 622 that may be used to initiate the application of the stimulation patterns to the thermodes. The stimulation patterns should be started at approximately the same time that image capture sequence of the MRI system is begun in order to be able to associate observed activity with the stimulation pattern. It is contemplated that the application of the stimulation pattern could be initiated in coordination with the MRI system by connecting the control device 600 to a connection of the fMRI that provides an electrical signal when the image capture sequence is initiated. The device 600 may further comprise a plurality of light emitting diodes 624 or other display device or devices. The LEDs 624 may be used to display various information, such as if a thermode is currently in the on period and if the thermode is actively being heated.

The control device 600 has been described with regards to a control device for controlling thermodes. It will be appreciated that particular details of the control device may be adjusted based on the specific stimulation devices used. Further, although the control device has been described as including a microcontroller for controlling the thermodes in accordance with the stimulation pattern, it is contemplated that the output to the thermodes could be controlled by another processor, such as the processor of the image analysis device described above with reference to FIG. 1, or a processor of the MRI system.

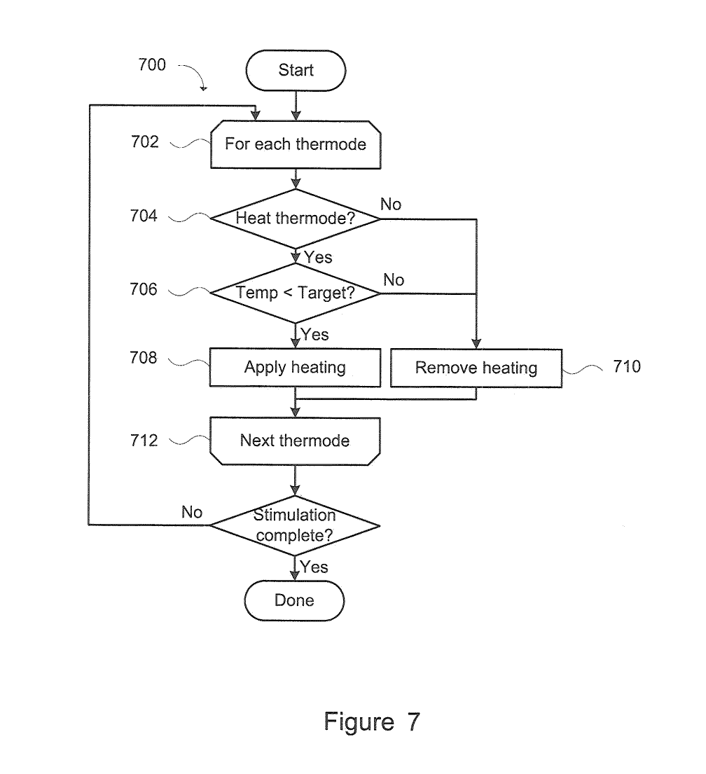

FIG. 7 depicts in a flow diagram a method of controlling stimulation of a plurality of thermodes. The method may be implemented by the microcontroller of the device described above. The method 700 controls the heating of each of the thermodes in accordance with the stimulation pattern, which specifies when a particular thermode should be heated. For each of the thermodes (702), the method checks to determine if the thermode should be heated (704) at this time. If the thermode is to be heated (Yes at 704), the method determines if the temperature of the thermode is less than the target temperature (706). If the temperature is less than the target temperature (Yes at 706) the method applies heating to the thermode (708). As described above, the application of heating to a thermode may be controlled by a microcontroller through an appropriate control signal which may, for example close an associated relay to connect the heating element of the thermode to a power source. If the thermode is not to be heated at this time (No at 704) or if the temperature is equal to or above the target temperature (No at 706), the method removes heating to the thermode (710). Hysteresis may be used when applying or removing heating in order to avoid quickly applying and removing the heating, that is the heating may be applied until the temperature has increased above an upper threshold, and may not be applied again until the temperature has fallen below a lower threshold. Although described as removing the heating, it will be appreciated that the controller may attempt to remove the heating, for example by opening the associated relay, even if the heating has not yet been applied.

Once the heating has been applied or removed from the thermode, the method repeats the process for the next thermode (712). Once all of the thermodes have been processed, the method determines if the stimulation is complete (712), and if it is (Yes at 712) the method is complete. If the stimulation is not complete (No at 712) the method returns to process each of the thermodes (702) again.

The thermodes apply the stimulation to the associated dermatones of the patient. While the stimulation pattern is being applied, a time series of images are captured by the MRI system, which can be subsequently processed. Since the application of the stimulation pattern and the image capture sequence are initiated at approximately the same time, each captured image can be associated with a specific time in the stimulation patterns. The response observed in the images can be matched to one of the stimulation patterns, allowing observed activity to be associated with one of the stimulation devices connected to a particular dermatone. Although the stimulation of individual stimulation devices may overlap in time, the stimulation patterns are linearly independent and so an observed response can be matched to a single stimulation pattern and associated stimulation device. As a result, the activity associated with each individual stimulation device can be identified in the captured images.

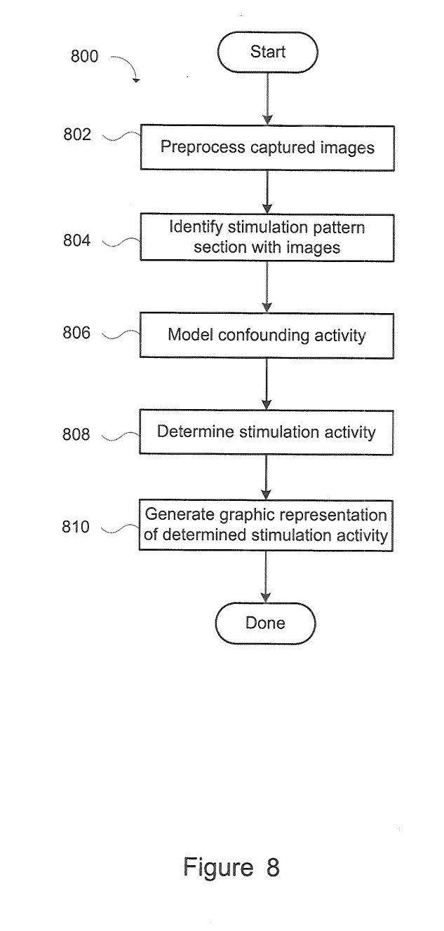

FIG. 8 depicts in a flow diagram a method of analyzing images to assess spinal cord function. The method 800 may be implemented in an image analysis device that processes images captured by the MRI system. The images may be supplied to the image analysis device in various ways. For example, the image analysis device may be coupled to the MRI system, or a computer controlling the MRI system through a communication network over which the images may be transferred. Additionally or alternatively, the images may be stored to a removable media by the MRI system and the removable media, including the captured images may be transferred to image analysis device. The method 800 preprocesses the received images (802). The preprocessing may comprise artifact removal, filtering and smoothing of the received images. The preprocessing of the images may further involve motion correction to correct for any movement of the patient during the capturing of the images. Once the images are preprocessed, a section of the stimulation pattern is identified (804) during which the image was captured. By identifying a section of the stimulation pattern associated with each of the captured images, the activity level of the spinal cord function can be associated with the stimulation level of the various stimulation devices. Confounding activity may be modeled (806) to account for activity not associated with the stimulation pattern applied to the thermodes. The confound modeling may account for confounding signals providing other sources of signal intensity change that are not a result of neural activity. Once the confounding activity is modeled, the activity due to the stimulation can be determined (808) for each of the captured images. The stimulation activity levels can be associated with a specific stimulation pattern and so stimulation device attached to a dermatone, allowing the spinal cord activity both above and below an area being assessed to be observed. The modeled confounds may be included as another component of signal change when trying to fit the stimulation pattern to the measured signal intensity.

Once the stimulation activity associated with each stimulation device is determined, a graphic representation can be generated (810). The graphic representation of the stimulation activity can be displayed in various ways. For example, the stimulation activity levels of each stimulation devices may be indicated by providing a color overlay on the captured images. The color overlay can allow the location and level of activity associated with each stimulated dermatone to be depicted. For example, each dermatone can be associated with a respective color, and the location of spinal cord function activity associated with the stimulation of the particular dermatone can be depicted by the overlaying of the respective color over one or more of the captured images, or a representation of the captured images, at the location of the activity. Further the brightness of the color may be representative of the activity level.

It will be appreciated that the graphic representation may be provided in numerous ways. Additionally, the graphic representation may be interactive to allow the information depicted to be adjusted by a user. For example, the activity associated with stimulation devices to display may be selected.

The system, device and methods described above provide the ability to stimulate a plurality of stimulation devices and analyze a time series of images captured by an MRI system during the stimulation in order assess spinal cord function. The system, device and methods described herein have been described with reference to various examples. It will be appreciated that components from the various examples may be combined together, or components of the examples removed or modified. As described the system may be implemented in one or more hardware components including a processing unit and a memory unit that are configured to provide the functionality as described herein. Furthermore, a computer readable memory, such as for example electronic memory devices, magnetic memory devices and/or optical memory devices, may store computer readable instructions for configuring one or more hardware components to provide the functionality described herein.

What is claimed is:

1. A method of assessing spinal cord function comprising:
applying a plurality of linearly independent stimulation patterns to a plurality of stimulation devices, each of the stimulation patterns applied to a respective stimulation device attached to a respective dermatome of a patient;
capturing a plurality of functional magnetic resonance (MR) images during the application of the plurality of linearly independent stimulation patterns; and
analyzing the plurality of captured functional MR images based on the applied linearly independent stimulation patterns to assess spinal cord function.

2. The method of claim 1, wherein analyzing the plurality of functional MR images comprises processing the functional MR images to identify neural activity from dermatones stimulated in accordance with the stimulation pattern.

3. The method of claim 1, wherein analyzing the plurality of functional MR images further comprises pre-processing the images to compensate for motion between captured functional MR images.

4. The method of claim 1, wherein analyzing the plurality of functional MR images further comprises defining features in the captured functional MR images corresponding to anatomical features.

5. The method of claim 1, wherein analyzing the plurality of functional MR images further comprises confound modelling to model signal intensity changes not of interest.

6. The method of claim 1, wherein the stimulation devices comprise thermodes and wherein applying the stimulation pattern comprises heating and cooling each thermode.

7. The method of claim 1, wherein applying the stimulation pattern to the plurality of stimulation devices comprises applying a base stimulation pattern to each stimulation device.

8. The method of claim 7, the base stimulation pattern comprises an on period, during which the thermode is heated and maintained at a specific temperature, followed by an off period, during which the thermode is cooled.

9. The method of claim 8, wherein the on period lasts for in the range of 45 seconds and the off period lasts for in the range of 105 seconds.

10. A system for assessing spinal cord function comprising:
a plurality of stimulation devices for attachment to respective dermatomes of a patient;
a control device for applying a plurality of linearly independent stimulation patterns to a plurality of stimulation devices, each of the stimulation patterns applied to a respective stimulation device and
an analysis device for analyzing a plurality of functional magnetic resonance (MR) images of the patient captured during the application of the stimulation patterns to assess spinal cord function, the analysis of the plurality of functional MR images based on the applied linearly independent stimulation patterns.

11. The system of claim 10, wherein at least one of the stimulation devices comprises a thermode having a heating element and a temperature sensing element.

12. The system of claim 11, wherein the heating element comprises one or more of:
a resistor; and
a peltier device.

13. The system of claim 11, wherein the temperature sensing element comprises a thermistor.

14. The system of claim of 10, wherein the control device comprises a microcontroller for applying the stimulation pattern to the stimulation devices.

15. The system of claim 11, wherein the control device comprises a plurality of relays, each associated with a respective one of the plurality of stimulation devices for selectively applying a voltage across the heating element of the stimulation device.

16. The system of claim 10, wherein the control device comprises an activation trigger to start the application of the stimulation pattern.

17. A control device for use in assessing spinal cord function comprising:
an interface for connecting the control device to a plurality of stimulation devices;
a microcontroller for applying a respective stimulation pattern of a plurality of linearly independent stimulation patterns to a respective stimulation device when connected to the interface.

18. The control device of claim 17, further comprising a plurality of relays, each associated with a respective one of the plurality of stimulation devices for selectively applying a voltage across the heating element of the stimulation device.

19. The control device of claim 17, further comprising an activation trigger to start the application of the stimulation patterns.

20. A kit comprising:
a control device for use in assessing spinal cord function comprising:
an interface for connecting the control device to a plurality of stimulation devices;
a microcontroller for applying a plurality of linearly independent stimulation patterns to a plurality of stimulation devices, each of the stimulation patterns applied to a respective stimulation device when connected to the interface; and
a plurality of stimulation devices for attaching to a patient and connecting to the device interface of the control device.
